(19) United States
(12) Patent Application Publication
(10) Pub. No.: US 2024/0058241 A1
(43) Pub. Date: Feb. 22, 2024

(54) SKIN WHITENING COMPOSITION

(71) Applicant: Richtrong Group LLC, Lewes, DE (US)

(72) Inventors: Tsong-Min CHANG, Taichung City (TW); Shr-Shiuan WANG, Taichung City (TW); Yi-Chang LI, Taichung City (TW); Huey-Chun HUANG, Taichung City (TW); Ya-Hui CHANG, New Taipei City (TW); Hui-Hsin YANG, New Taipei City (TW)

(21) Appl. No.: 18/156,534

(22) Filed: Jan. 19, 2023

(30) Foreign Application Priority Data
Aug. 22, 2022 (TW) 111131453

(51) Int. Cl. A61K 8/34; A61K 8/60; A61Q 19/02

(52) U.S. Cl. CPC A61K 8/347; A61K 8/606; A61Q 19/02; A61K 2800/805

(57) ABSTRACT

Disclosed herein is a skin whitening composition that includes, based on a total volume of the skin whitening composition, 0.5 μM to 10 μM of resveratrol, and 2.5 μM to 500 μM of nicotinamide mononucleotide.

### CROSS-REFERENCE TO RELATED APPLICATION

This application claims priority of Taiwanese Invention Patent Application No. 111131453, filed on Aug. 22, 2022.

### FIELD

The present disclosure relates to a skin whitening composition including 0.5 μM to 10 μM of resveratrol, and 2.5 μM to 500 μM of nicotinamide mononucleotide, based on the total volume of the skin whitening composition.

### BACKGROUND

At present, consumers attach great importance to skin care, in particular, how to keep the skin bright and white has become a topic of concern for many consumers. Nicotinamide mononucleotide (NMN) is a type of bioactive nucleotide which is naturally formed by the reaction between a phosphate group and a nucleoside containing ribose and nicotinamide. NMN is naturally found in certain whole foods (such as edamame, tomato, cabbage, cucumber, and avocado). It is known that NMN can effectively inhibit melanin production, and hence is widely used in skin whitening products. In the cosmetic industry, the use of NMN obtained from natural foods to prepare skin whitening products is more desirable and safer, but might result in a high production cost.

In spite of the aforesaid, there is still a need for those skilled in the art to develop a skin whitening composition having excellent tyrosinase inhibitory effect and low production cost, so as to meet industrial requirements.

### SUMMARY

Therefore, an object of the present disclosure is to provide a skin whitening composition that can alleviate at least one of the drawbacks of the prior art.

The skin whitening composition includes, based on a total volume of the skin whitening composition: 0.5 μM to 10 μM of resveratrol, and 2.5 μM to 500 μM of nicotinamide mononucleotide.

### DETAILED DESCRIPTION

For the purpose of this specification, it will be clearly understood that the word “comprising” means “including but not limited to”, and that the word “comprises” has a corresponding meaning.

It is to be understood that, if any prior art publication is referred to herein, such reference does not constitute an admission that the publication forms a part of the common general knowledge in the art, in Taiwan or any other country.

Unless defined otherwise, all technical and scientific terms used herein have the meaning commonly understood by a person skilled in the art to which the present disclosure belongs. One skilled in the art will recognize many methods and materials similar or equivalent to those described herein, which could be used in the practice of the present disclosure. Indeed, the present disclosure is in no way limited to the methods and materials described.

The present disclosure provides a skin whitening composition including, based on a total volume of the skin whitening composition: 0.5 μM to 10 μM of resveratrol, and 2.5 μM to 500 μM of nicotinamide mononucleotide. The skin whitening composition can inhibit the activity of tyrosinase, and hence is capable of inhibiting melanin production.

In certain embodiments, resveratrol may be present in a concentration ranging from 1 μM to 10 μM, based on the total volume of the skin whitening composition.

In certain embodiments, nicotinamide mononucleotide may be present in a concentration ranging from 2.5 μM to 187.5 μM, based on the total volume of the skin whitening composition. In an exemplary embodiment, nicotinamide mononucleotide is present in a concentration ranging from 2.5 μM to 175 μM, based on the total volume of the skin whitening composition.

In certain embodiments, resveratrol may be dissolved in a solvent selected from the group consisting of ethanol, dimethyl sulfoxide (DMSO), water, and phosphate-buffered saline (PBS) (pH 7.2). In an exemplary embodiment, resveratrol is dissolved in ethanol.

In certain embodiments, nicotinamide mononucleotide may be dissolved in a solvent selected from the group consisting of double distilled water (ddH₂O) and methanol. In an exemplary embodiment, nicotinamide mononucleotide is dissolved in ddH₂O.

According to the present disclosure, the skin whitening composition may be formulated into an external preparation suitable for topical application to the skin using technology well known to those skilled in the art. The external preparation includes, but is not limited to, emulsions, gels, ointments, creams, patches, liniments, powder, aerosols, sprays, lotions, serums, pastes, foams, drops, suspensions, salves, and bandages.

According to the present disclosure, the external preparation is prepared by admixing the skin whitening composition with a base that is well known and commonly used in the art.

According to the present disclosure, the base may include one or more of the following additives: water, alcohols, glycols, hydrocarbons (such as petroleum jelly and white petrolatum), waxes (such as paraffin and yellow wax), preserving agents, antioxidants, surfactants, absorption enhancers, stabilizing agents, gelling agents (such as Carbopol®941, microcrystalline cellulose and carboxymethylcellulose), active agents, humectants, odor absorbers, fragrances, pH-adjusting agents, chelating agents, emulsifiers, occlusive agents, emollients, thickeners, solubilizing agents, penetration enhancers, anti-irritants, colorants, propellants, etc. The choice and amount of the aforesaid additives are within the expertise and the routine skills of those skilled in the art.

The dosage and the frequency of administration of the skin whitening composition according to the present disclosure may vary depending on the following factors: the severity of the disease to be treated, the route of administration, and the weight, age, physical condition and response of the subject to be treated. The daily dosage of the skin whitening composition according to the present disclosure may be administered in a single dose or in several doses.

The disclosure will be further described by way of the following examples. However, it should be understood that the following examples are solely intended for the purpose of illustration and should not be construed as limiting the disclosure in practice.

### EXAMPLES

### Preparation of Resveratrol Stock Solution:

0.457 mg of resveratrol was mixed with 10 ml 99% (w/w) ethanol, so as to obtain a resveratrol stock solution having a concentration of 0.2 mM. The resveratrol stock solution was used in the following experiments.

### Preparation of Nicotinamide Mononucleotide Stock Solution:

100 mg of nicotinamide mononucleotide was mixed with 5.98 ml double distilled water (ddH₂O), so as to obtain a nicotinamide mononucleotide stock solution having a concentration of 50 mM. The nicotinamide mononucleotide stock solution was used in the following experiments.

### Example 1 (EX1)

2.5 μL of the resveratrol stock solution was mixed with 5 μL of the nicotinamide mononucleotide stock solution and 0.9925 ml of water, so as to obtain a skin whitening composition containing 0.5 μM resveratrol and 2.5 μM nicotinamide mononucleotide.

### Examples 2 to 16 (EX2 to EX18)

The procedures for preparing the skin whitening compositions of EX2 to EX18 were similar to those of EX1, except that the concentrations of resveratrol and nicotinamide mononucleotide were varied as shown in Table 1 below.

### Comparative Example 1 (CE1)

5 μL of the nicotinamide mononucleotide stock solution was mixed with 0.995 ml of water, so as to obtain a nicotinamide mononucleotide solution having a concentration of 2.5 μM.

### Comparative Examples 2 to 6 (CE2 to CE6) and 12 (CE12)

The procedures for preparing the nicotinamide mononucleotide solutions of CE2 to CE6 and CE12 were similar to those of CE1, except that the concentration of nicotinamide mononucleotide was varied as shown in Table 2 below.

### Comparative Example 7 (CE7)

2.5 μL of the resveratrol stock solution was mixed with 0.9975 ml of water, so as to obtain a resveratrol solution having a concentration of 0.5 μM.

### Comparative Examples 8 to 11 (CE8 to CE11) and 13 to 14 (CE13 to CE14)

The procedures for preparing the resveratrol solutions of CE8 to CE11 and CE13 to CE14 were similar to those of CE7, except that the concentration of resveratrol was varied as shown in Table 2 below.

TABLE 1

| | | Nicotinamide |
|---|---|---|
| | Resveratrol | mononucleotide |
| EX | (μM) | (μM) |
| 1 | 0.5 | 2.5 |
| 2 | 0.5 | 25 |
| 3 | 0.5 | 50 |
| 4 | 0.5 | 75 |
| 5 | 0.5 | 175 |
| 6 | 0.5 | 187.5 |
| 7 | 1 | 2.5 |
| 8 | 1 | 25 |
| 9 | 1 | 50 |
| 10 | 1 | 75 |
| 11 | 1 | 175 |
| 12 | 1 | 187.5 |
| 13 | 10 | 25 |
| 14 | 10 | 75 |
| 15 | 10 | 175 |
| 16 | 10 | 187.5 |
| 17 | 1 | 500 |
| 18 | 2 | 500 |

TABLE 2

| | | Nicotinamide |
|---|---|---|
| | Resveratrol | mononucleotide |
| CE | (μM) | (μM) |
| 1 | 0 | 2.5 |
| 2 | 0 | 25 |
| 3 | 0 | 50 |
| 4 | 0 | 75 |
| 5 | 0 | 175 |
| 6 | 0 | 187.5 |
| 7 | 0.5 | 0 |
| 8 | 1 | 0 |
| 9 | 3 | 0 |
| 10 | 5 | 0 |
| 11 | 10 | 0 |
| 12 | 0 | 500 |
| 13 | 1 | 0 |
| 14 | 2 | 0 |

### Property Evaluation:

### A. Tyrosinase Inhibition Assay

78 μL of the skin whitening composition of EX1 was mixed with 2 μL of a tyrosinase solution (containing 1 U/μL tyrosinase) and 120 μL of a L-DOPA solution (containing 5 mM L-DOPA and 67 mM phosphate-buffered saline), followed by being left standing for reaction to proceed at 37° C. for 30 minutes, so as to obtain a first test solution. In addition, 2 μL of a tyrosinase solution (containing 1 U/μL tyrosinase) was mixed with 120 μL of a L-DOPA solution (containing 5 mM L-DOPA and 67 mM phosphate-buffered saline), followed by being left standing for reaction to proceed at 37° C. for 30 minutes, so as to obtain a second test solution.

The respective resultant test solution was subjected to determination of absorbance at a wavelength of 490 nm (OD₄₉₀) by using a microplate fluorometer (Manufacturer: Thermo Scientific; Model No.: Fluoroskan Ascent 52104710). All the experiments described above were performed in triplicates. The experimental data (i.e., OD₄₉₀ value) of the respective test solution are expressed as mean.

The tyrosinase inhibition rate (%) was calculated using the following Equation (I):

A=[(B−C)/B]×100 (I)
where A=tyrosinase inhibition rate (%)
   B=OD₄₉₀ value of the second test solution
   C=OD₄₉₀ value of the first test solution

The skin whitening compositions of EX2 to EX16, the nicotinamide mononucleotide solutions of CE1 to CE6, and the resveratrol solutions of CE7 to CE11 were subjected to the same reaction and measurement as described above.

The results are shown in Table 3 below.

### B. Determination of Relative Tyrosinase Content

Murine melanoma cell line B16F10 (BCRC 60031) used in the following experiments was purchased from the Bioresource Collection and Research Center (BCRC) of the Food Industry Research and Development Institute (FIRDI) (No. 331, Shih-Pin Rd., Hsinchu City 300, Taiwan).

The B16F10 cells were incubated in a respective well of a 24-well culture plate containing 1 mL of Dulbecco's modified Eagle's medium (DMEM) (high glucose) supplemented with 100 nM α-melanocyte-stimulating hormone (α-MSH) at 5×10⁴ cells/well, followed by cultivation in an incubator (37° C., 5% CO2) for 24 hours. Next, each of the cell cultures was treated with 1 mL of the skin whitening composition of EX17, followed by cultivation in an incubator (37° C., 5% CO2) for 24 hours. After that, the liquid in each well was removed, and the cells were washed twice with phosphate-buffered saline (PBS), and then were detached from the respective well.

The cells were harvested and then mixed with 100 μL of a 1% (v/v) treating solution (containing 4-(1,1,3,3-tetramethylbutyl)phenyl-polyethylene glycol (Triton™ X-100), 10 mM phenylmethanesulfonyl fluoride (PMSF), and 50 mM PBS). The resultant mixture was froze in a refrigerator set at −80° C. for 30 minutes, followed by thawing at 25° C. for 30 minutes. The above freeze-thaw treatment was performed 2 to 3 times. Then, the resultant thawed solution was subjected to centrifugation at 4° C. under a speed of 12,000 rpm for 30 minutes so as to form supernatant and pellet fractions. After that, 80 μL of the supernatant and 40 μL of a L-DOPA solution (containing 5 mM L-DOPA and 67 mM phosphate-buffered saline) were added in a respective well of a 96-well culture plate, followed by being left standing for reaction to proceed at 37° C. for 1 hour, so as to obtain a test sample.

In addition, a control sample was prepared according to the procedure described above, except that the B16F10 cells were not treated with the skin whitening composition of EX17.

A respective one of the test sample and the control sample was subjected to determination of absorbance at a wavelength of 490 nm (OD₄₉₀) by using a microplate fluorometer (Manufacturer: Thermo Scientific; Model No.: Fluoroskan Ascent 52104710). All the experiments described above were performed in quadruplicate. The experimental data (i.e., OD₄₉₀ value) of the respective sample are expressed as mean.

The relative tyrosinase content (%) was calculated using the following Equation (II):

D=(E/F)×100 (II)
where D=relative tyrosinase content (%)
   E=OD₄₉₀ value of the test sample
   F=OD₄₉₀ value of the control sample

The skin whitening composition of EX18, the nicotinamide mononucleotide solution of CE12, and the resveratrol solutions of CE13 to CE14 were subjected to the same reaction and measurement as described above. The results are shown in Table 4 below.

### C. Determination of Relative Melanin Content

The B16F10 cells described in section B were incubated in a respective well of a 24-well culture plate containing 1 mL of DMEM (high glucose) supplemented with 100 nM α-MSH at 5×10⁴ cells/well, followed by cultivation in an incubator (37° C., 5% 002) for 24 hours. Next, each of the cell cultures was treated with 1 mL of the skin whitening composition of EX17, followed by cultivation in an incubator (37° C., 5% 002) for 24 hours. After that, the liquid in each well was removed, and the cells were washed twice with phosphate-buffered saline (PBS), and then were detached from the respective well.

The cells were harvested, and then were mixed with 100 μL of a 1 N sodium hydroxide solution, followed by being left standing at 60° C. for 1 hour, so as to obtain a test sample. In addition, a control sample was prepared according to the procedure described above, except that the B16F10 cells were not treated with the skin whitening composition of EX17.

A respective one of the test sample and the control sample was subjected to determination of absorbance at a wavelength of 405 nm (OD₄₀₅) by using a microplate fluorometer (Manufacturer: Thermo Scientific; Model No.: Fluoroskan Ascent 52104710). All the experiments described above were performed in quadruplicate. The experimental data (i.e., OD₄₀₅ value) of the respective sample are expressed as mean.

The relative melanin content (%) was calculated using the following Equation (III):

G=(H/I)×100 (III)
where G=relative melanin content (%)
   H=OD₄₀₅ value of the test sample
   I=OD₄₀₅ value of the control sample

The skin whitening composition of EX18, the nicotinamide mononucleotide solution of CE12, and the resveratrol solutions of CE13 to CE14 were subjected to the same reaction and measurement as described above. The results are shown in Table 4 below.

### Results:

As shown in Table 3, the tyrosinase inhibition rates determined in EX1 to EX6 were respectively higher than those determined in CE1 to CE6, indicating that by virtue of 0.5 μM resveratrol in combination with different concentrations of nicotinamide mononucleotide, the tyrosinase inhibition rates of EX1 to EX6 were increased by 12.18% (i.e., 108.41% minus 96.23%) to 50.75% (i.e., 59.59% minus 8.84%).

In addition, the tyrosinase inhibition rates determined in EX7 to EX12 were respectively higher than those determined in CE1 to CE6, indicating that by virtue of 1 μM resveratrol in combination with different concentrations of nicotinamide mononucleotide, the tyrosinase inhibition rates of EX7 to EX12 were increased by 4.23% (i.e., 100.46% minus 96.23%) to 62.27% (i.e., 71.11% minus 8.84%).

Moreover, the tyrosinase inhibition rates determined in EX13 to EX16 were respectively higher than those determined in CE2 and CE4 to CE6, indicating that by virtue of 10 μM resveratrol in combination with different concentrations of nicotinamide mononucleotide, the tyrosinase inhibition rates of EX13 to EX16 were increased by 20.21% (i.e., 116.44% minus 96.23%) to 82.41% (i.e., 91.25% minus 8.84%).

As shown in Table 4, the relative tyrosinase contents determined in EX17 and EX18 were respectively lower than that determined in CE12, indicating that by virtue of different concentrations (i.e., 1 μM and 2 μM) of resveratrol in combination with nicotinamide mononucleotide, the relative tyrosinase contents of EX17 and EX18 were reduced by 32.84% (i.e., 95.86% minus 63.02%) to 53.71% (i.e., 95.86% minus 42.15%).

In addition, the relative melanin contents determined in EX17 and EX18 were respectively lower than that determined in CE12, indicating that by virtue of different concentrations (i.e., 1 μM and 2 μM) of resveratrol in combination with nicotinamide mononucleotide, the relative melanin contents of EX17 and EX18 were reduced by 9.38% (i.e., 88.16% minus 78.78%) to 14.65% (i.e., 88.16% minus 73.51%).

Furthermore, the tyrosinase inhibition rate determined in EX7 was similar to that determined in CE4, indicating that by virtue of the combination of resveratrol and nicotinamide mononucleotide, the skin whitening composition of the present disclosure requires only 2.5 μM of nicotinamide mononucleotide (that is, the concentration of nicotinamide mononucleotide is reduced by a factor of 30) to achieve a similar effect.

The skin whitening composition of CE5 required 175 μM of nicotinamide mononucleotide to achieve a tyrosinase inhibition rate of 69.33%. On the contrary, by virtue of the combination of resveratrol and nicotinamide mononucleotide, the skin whitening compositions of EX3 and EX8 required lower nicotinamide mononucleotide content (i.e., only 50 μM and 25 μM, respectively) to achieve a relatively high tyrosinase inhibition rate (i.e., 74.83% and 71.11%, respectively).

TABLE 3

| | | Tyrosinase inhibition rate (%) |
|---|---|---|
| EX | 1 | 20.95 |
| | 2 | 59.59 |
| | 3 | 74.83 |
| | 4 | 94.22 |
| | 5 | 98.45 |
| | 6 | 108.41 |
| | 7 | 44.06 |
| | 8 | 71.11 |
| | 9 | 93.48 |
| | 10 | 98.75 |
| | 11 | 104.84 |
| | 12 | 100.46 |
| | 13 | 91.25 |
| | 14 | 111.76 |
| | 15 | 119.48 |
| | 16 | 116.44 |
| CE | 1 | 6.69 |
| | 2 | 8.84 |
| | 3 | 34.11 |
| | 4 | 46.81 |
| | 5 | 69.33 |
| | 6 | 96.23 |
| | 7 | 11.29 |
| | 8 | 29.50 |
| | 9 | 53.20 |
| | 10 | 59.73 |
| | 11 | 59.74 |

TABLE 4

| | | Relative tyrosinase content | Relative melanin content |
|---|---|---|---|
| | | (%) | (%) |
| EX | 17 | 63.02 | 78.78 |
| | 18 | 42.15 | 73.51 |
| CE | 12 | 95.86 | 88.16 |
| | 13 | 79.76 | 86.64 |
| | 14 | 74.52 | 79.95 |

Summarizing the above test results, it is clear that by virtue of different concentrations (i.e., 0.5 μM, 1 μM, 2 μM, and 10 μM) of resveratrol in combination with different concentrations (i.e., 2.5 μM, 25 μM, 50 μM, 75 μM, 175 μM, 187.5 μM, and 500 μM) of nicotinamide mononucleotide, the skin whitening composition of the present disclosure is capable of improving tyrosinase inhibition rate and reducing relative tyrosinase content and relative melanin content, and hence can effectively inhibit the formation of melanin and can exhibit excellent skin whitening effect.

In the description above, for the purposes of explanation, numerous specific details have been set forth in order to provide a thorough understanding of the embodiment(s). It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects; such does not mean that every one of these features needs to be practiced with the presence of all the other features. In other words, in any described embodiment, when implementation of one or more features or specific details does not affect implementation of another one or more features or specific details, said one or more features may be singled out and practiced alone without said another one or more features or specific details. It should be further noted that one or more features or specific details from one embodiment may be practiced together with one or more features or specific details from another embodiment, where appropriate, in the practice of the disclosure.

While the disclosure has been described in connection with what is (are) considered the exemplary embodiment(s), it is understood that this disclosure is not limited to the disclosed embodiment(s) but is intended to cover various arrangements included within the spirit and scope of the broadest interpretation so as to encompass all such modifications and equivalent arrangements.

What is claimed is:

1. A skin whitening composition comprising, based on a total volume of the skin whitening composition:
0.5 μM to 10 μM of resveratrol; and
2.5 μM to 500 μM of nicotinamide mononucleotide.

2. The skin whitening composition as claimed in claim 1, wherein resveratrol is present in a concentration ranging from 1 μM to 10 μM, based on the total volume of the skin whitening composition.

3. The skin whitening composition as claimed in claim 1, wherein nicotinamide mononucleotide is present in a concentration ranging from 2.5 μM to 187.5 μM, based on the total volume of the skin whitening composition.

4. The skin whitening composition as claimed in claim 3, wherein nicotinamide mononucleotide is present in a concentration ranging from 2.5 μM to 175 μM, based on the total volume of the skin whitening composition.

5. The skin whitening composition as claimed in claim 1, wherein resveratrol is dissolved in a solvent selected from the group consisting of ethanol, dimethyl sulfoxide (DMSO), water, and phosphate-buffered saline (PBS) (pH 7.2).

6. The skin whitening composition as claimed in claim 1, wherein nicotinamide mononucleotide is dissolved in a solvent selected from the group consisting of double distilled water (ddH₂O) and methanol.
